(19) United States
(12) Patent Application Publication
(10) Pub. No.: US 2024/0389976 A1
(43) Pub. Date: Nov. 28, 2024

(54) SYSTEMS, DEVICES AND METHODS FOR ULTRASOUND DETECTION OF VASCULAR HEMODYNAMIC MEASURES

(71) Applicant: MOONRISE MEDICAL, INC., OAKVILLE (CA)

(72) Inventors: ADAM GOLD, SCOTTSDALE, AZ (US); ABUBAKER KHALIFA, MILTON (CA); JILL SOMMERSET, PARKDALE, OR (US); JEFFREY LEADBETTER, HALIFAX (CA); JULIAN ZOU, HALIFAX (CA); JEREMY BROWN, HALIFAX (CA)

(21) Appl. No.: 18/693,309

(22) PCT Filed: Mar. 28, 2023
(86) PCT No.: PCT/CA2023/050413

§ 371 Date: Mar. 19, 2024

Related U.S. Application Data
(60) Provisional application No. 63/624,328, filed on Jan. 24, 2024.

(51) Int. Cl. A61B 8/00; A61B 8/06; A61B 8/08

(52) U.S. Cl. CPC A61B 8/4236; A61B 8/06; A61B 8/4245; A61B 8/429; A61B 8/4488; A61B 8/488

(57) ABSTRACT

Ultrasound systems and devices are provided that facilitate positioning and orientation of an ultrasound beam relative to the vasculature of the patient for the detection of Doppler signals from vessels and the determination of a hemodynamic measure. In some example embodiments, a support structure, such as a patch, is provided that supports one or more ultrasound transducers relative to a coupling component, the support structure being removably attachable to a skin surface of a subject. Control and processing circuitry is connectable to the one or more ultrasound transducers to generate ultrasound beams, receive ultrasound signals, and process the received ultrasound signals to provide a Doppler signal and determine an associated hemodynamic measure. In some example implementations, the example embodiments disclosed herein may be employed to determine a hemodynamic measure associated with blood flow with major and/or minor vessels of the pedal arch, such as a pedal acceleration time.

### CROSS-REFERENCE TO RELATED APPLICATION

This application claims priority to U.S. Provisional Patent Application No. 63/324,328, titled “SYSTEMS, DEVICES AND METHODS FOR ULTRASOUND DETECTION OF VASCULAR HEMODYNAMIC MEASURES” and filed on Mar. 28, 2022 the entire contents of which is incorporated herein by reference.

### BACKGROUND

The present disclosure relates to the detection of vascular disorders using ultrasound.

Peripheral arterial disease (PAD) is caused by a narrowing or blockage of the blood vessels (arteries) that carry blood from the heart to the rest of your body. PAD is most commonly caused by a build-up of plaque that reduces blood flow to the legs and causes foot pain and ulcers that do not heal. It could even mean complete loss of blood circulation and can result in gangrene and amputation of the affected body part if it goes untreated. Chronic limb threatening ischemia (CLTI) is a severe form of PAD. Patients with CLTI often have chronic pain, even at rest, as well as ulcers and gangrene (tissue death) that develops as a result of long-term poor blood flow to the lower limbs.

There are numerous methods and technologies available to diagnose and classify PAD and CLTI. One common test, the Ankle-Brachial Index (ABI), compares the blood pressure measured at the ankle with the blood pressure measured at the arm. Another common test, the Toe-Brachial Index (TBI), measures the blood pressure measured at the toe with the blood pressure measured at the arm. While these tests are commonly used, they can be unreliable when the blood vessels are heavily calcified, as is the case in many PAD patients with diabetes. Furthermore, ankle-level pressures and waveforms may incompletely characterize the extent of disease in those with isolated infra-popliteal and infra-maleloar disease. In the setting of extensive wounds involving the toes or prior forefoot amputation, toe waveforms and/or pressures may not obtained.

Computed tomography angiography (CTA) and magnetic resonance angiography (MRA) are also limited by arterial wall calcification. Such tests are not easily integrated to a clinic outpatient setting or in the intraoperative suite. Transcutaneous oxygen saturation testing (TCPO2) and skin perfusion pressure testing (SPP) also have limitations such as false readings in the setting of significant tissue edema or in the presence of large wounds, which can interfere with the proper placement of cuffs and probes. Indocyanine green angiography (ICGA) has also been used in evaluating tissue perfusion. However, it is invasive in nature and requires additional capital expenditure. It is also relatively subjective and not readily portable.

Optical technologies, such as those described in U.S. Pat. No. 10,213,122 (Lee et al.) and United States Patent Publication No. 2018/0199829 (White et al.) evaluate perfusion only millimeters below the skin surface and cannot evaluate the vessels of the pedal arch that reside 1-2 cm below the skin surface. Additionally, these sensors detect are focused on the microvasculature below the skin surface. If there is severe small vessel disease, the measurement may not be reliable. Also, these optical techniques in principle require a coherent optical light source and another detector to indirectly determine flow. This could be impacted by the presence of edema, patient movement, wounds, small microvascular disease commonly seen in PAD patients with diabetes. If the patient has a surgically absent digit, which is often required, then this test will be limited in its current form.

In light of the above technological limitations, there is a need for a device to reliably and inexpensively diagnose tissue perfusion of larger, calcified vessels of the extremities, particularly the pedal arch.

When PAD/CLTI is diagnosed, surgical intervention can often be attempted to open the proximal vessels so as to improve blood flow to a wound, promote wound healing, and avoid amputation. These procedures may be open surgery or endovascular. Typically, the interventionalist will use fluoroscopy with contrast to image the arteries in the limbs. They perform interventions, such as angioplasty (balloon), atherectomy, or stent to open a stenosis. After intervention, they use fluoroscopy with contrast again to determine how well their intervention improved distal perfusion. This technique is not ideal because fluoroscopy is not a quantitative measure of perfusion. Moreover, fluoroscopic readings are not necessarily predictive of desired outcomes, such as wound healing.

After the first intervention, the interventionalist is faced with the decision of whether to perform additional interventions. Additional interventions can be performed on the same vessel or additional vessels. While additional interventions may improve perfusion and outcomes, they carry additional risks and require additional time in the cath lab/OR. More importantly, interventionalists typically do not know which or how many interventions will lead to positive outcomes. In short, they do not know “how much is enough, or when to stop?” when performing interventions.

Devices such as those described in U.S. Pat. No. 1,023,122 (Lee, et al) and U.S. Pat. No. 20,180,199829A1 (White, et al) typically evaluate microvessel perfusion. This can be problematic in the intra-operative setting because there is often a delay between the time larger, more proximal vessels are opened and perfusion through the distal microvasculature due to swelling, edema, and other factors.

As such, there is a need for an intraoperative surgical planning tool that can analyze perfusion in the pedal vessels in real-time and is predictive of clinically accepted and relevant clinical outcomes such as wound healing and limb salvage.

FIGS. 1-3 for pedal anatomy, where FIG. 1 illustrates the major pedal arteries that comprise the posterior circulation of the foot, FIG. 2 illustrates the major pedal arteries that comprise the anterior circulation of the foot, and FIG. 3 illustrates the complete pedal arch, which is the connection between the anterior and posterior circulation of the foot.

In depth analysis of the pedal arch velocity waveform has shown that pedal acceleration time (PAT) is a critical variable that is directly relevant in determining the extent of disease severity in patients with CLTI, as described in Sommerset, J. in United States Patent Publication No. US 2020/0288993. More importantly, PAT has been discovered and studied by Sommerset to show that it is a strong predictor of clinical outcomes such as wound healing in patients with CLTI. The PAT method involves measuring the time between the onset of systole to the peak of systole on the arterial waveform obtained through ultrasound imaging. FIG. 4 shows a typical image taken from a vascular ultrasound. Imaging of the vessel with flow highlighted is shown on the top half. The velocity waveform of blood flow within the vessel is shown on the bottom of the screen. PAT is measured by the time C1 from point A1 to point B1. This displays the technique for proper and reliable measurement of acceleration time.

PAT classifications (FIG. 5) have been shown to correlate with ABI measurements, as well as predict wound healing. Therefore, since PAT is a quantifiable assessment of pedal perfusion in real-time, it is valuable for the interventionist to know when an adequate amount of perfusion has been reached. In contrast, microvascular measurements (such as laser speckle) often do not provide accurate real-time measurements because microvascular perfusion can lag due to inflammation and edema. PAT is particularly helpful when indirect revascularization is the only option in CLTI patients. Oftentimes the only option is to indirectly provide flow to the wound bed for optimal healing. PAT is helpful because blood flow can be monitored at the edge of the wound bed for precise evaluation of perfusion. In addition to intraoperative guidance, PAT can also be useful as a diagnostic tool to determine which patients can benefit from intervention, as well as postoperative surveillance to track patient's limb perfusion. This is highly relevant as pedal arch velocity waveform analysis, in particular PAT, can help screen and diagnose patients with PAD/CLTI. Most of these patients would not be accurately diagnosed as most of the current available technologies in the primary care and specialist clinics are unreliable in detecting PAD/CLTI and determining the overall prognosis. Post-operative surveillance of patients with PAD/CLTI is usually limited to subjective observation of the wound bed or patient symptomology, there are no reliable current diagnostic tools that are designed for this setting to help clinicals objectively determine the success of the operative procedure and predict short-term or long-term wound healing.

PAT is a non-invasive, low cost, high value study with the following features that contribute to the potential significance of this modality to the treatment of patients with peripheral arterial occlusive disease and CLTI: 1) Quantifiable: PAT provides a measurable value; it introduces a much-needed quantifiable indicator of arterial flow to the foot. 2) Established: PAT classification has already been demonstrated to show a strong correlation between clinical symptoms and ABIs (in patients in whom an ABI was obtainable and deemed reliable). 3) Predictive: an improvement in PAT post revascularization may be an indicator of limb salvage. 4) Sensitive and specific: PAT allows for the specific evaluation of the arterial flow to the foot in the setting of both indirect and direct angiosomic revascularization. 5) Anatomic site specific: unlike indirect tests such as TBIs and ABIs, PAT can be obtained at any pedal artery near or adjacent to the wound. Obtaining a PAT does not require the use of cumbersome pressure cuffs. 6) Safe: a non-invasive test performed by a registered vascular technologist at the bedside. 7) Portable and cost effective: there is no need to invest in expensive capital expenditure. Most available current models of duplex ultrasound have the capability of determining acceleration time and can be used in most patient settings. 8) Repeatable and accessible: PAT can be used in all phases of patient care: pre-operative, during the interventional case, and post-operative.

PAT has thus been shown to hold promise to support and improve the treatment and management of the complex CLTI patient.

### SUMMARY

Ultrasound systems and devices are provided that facilitate positioning and orientation of an ultrasound beam relative to the vasculature of the patient for the detection of Doppler signals from vessels and the determination of a hemodynamic measure. In some example embodiments, a support structure, such as a patch, is provided that supports one or more ultrasound transducers relative to a coupling component, the support structure being removably attachable to a skin surface of a subject. Control and processing circuitry is connectable to the one or more ultrasound transducers to generate ultrasound beams, receive ultrasound signals, and process the received ultrasound signals to provide a Doppler signal and determine an associated hemodynamic measure. In some example implementations, the example embodiments disclosed herein may be employed to determine a hemodynamic measure associated with blood flow with major and/or minor vessels of the pedal arch, such as a pedal acceleration time.

Accordingly, in a first aspect, there is provided an ultrasound device comprising:
a housing configured for attachment to a skin surface;
an ultrasound transducer assembly supported by the housing, the ultrasound transducer assembly comprising:
   an ultrasound transducer; and
   a coupling component for coupling ultrasound energy between the ultrasound transducer and the skin surface, wherein the coupling component is in acoustic communication with the ultrasound transducer, and wherein a distal portion of the coupling component is configured to contact and apply pressure to the skin surface when the housing is secured to the skin surface; and
wherein the ultrasound transducer assembly and the housing are configured such that the ultrasound transducer assembly is moveable, relative to the housing, for varying at least one of:
   an orientation of the ultrasound transducer assembly relative to the housing; and
   a lateral position of the ultrasound transducer assembly relative to the housing;
the ultrasound transducer assembly being supported, relative to the housing, such that a constant pressure is maintained between the distal portion of the coupling component and the skin surface when the ultrasound transducer assembly is moved relative to the housing.

In some example implementations of the ultrasound device, the housing is configured to permit rotation of the ultrasound transducer assembly about a rotation axis, and wherein a distal surface of the coupling component comprises a circular shape in a plane perpendicular to the rotation axis such that the constant pressure is maintained between the coupling component and the skin surface during rotation of the ultrasound transducer assembly.

In some example implementations of the ultrasound device, the housing comprises a socket, wherein at least a distal portion of the ultrasound transducer assembly is spherical in shape and resides within the socket, thereby forming a ball and socket joint, the socket comprising a distal aperture through which the ultrasound transducer assembly projects, such that the distal portion of the coupling component contacts and applies pressure to the skin surface when housing is secured to the skin surface, and such that the constant pressure is maintained between the coupling component and the skin surface during rotation of the ultrasound transducer assembly.

In some example implementations of the ultrasound device, the housing comprises a slot configured to permit lateral translation of the ultrasound transducer assembly relative to the housing in a direction parallel to the skin surface, and such that the constant pressure is maintained between the coupling component and the skin surface during lateral translation of the ultrasound transducer assembly.

In some example implementations of the ultrasound device, the housing is configured to permit lateral translation of the ultrasound transducer assembly relative to the housing in a translation direction that is parallel to the skin surface, and wherein the ultrasound transducer comprises a one-dimensional array of ultrasound elements, the one-dimensional array of ultrasound elements being disposed along a longitudinal axis that is parallel to the translation direction. The one-dimensional array of ultrasound elements may be angled relative to the skin surface.

In some example implementations of the ultrasound device, the housing is configured to generate a frictional force as a result of contact with the ultrasound transducer assembly, wherein the frictional force is sufficiently low to permit manual actuation thereof and sufficiently high to maintain the ultrasound transducer assembly in a fixed orientation in the absence of an external applied force.

In some example implementations of the ultrasound device, the housing is configured to facilitate variation of the orientation of the ultrasound transducer assembly relative to the housing, and wherein at least one the housing and the ultrasound transducer assembly comprises an orientation locking means for releasably locking the orientation of the ultrasound transducer relative to the housing.

In some example implementations of the ultrasound device, the housing is configured to facilitate variation of the lateral position of the ultrasound transducer assembly relative to the housing, and wherein at least one of the housing and the ultrasound transducer assembly comprises a position locking means for releasably locking the lateral position of the ultrasound transducer relative to the housing.

In some example implementations of the ultrasound device, the housing is configured to facilitate variation of the orientation of the ultrasound transducer assembly relative to the housing, and wherein at least one of the housing and the ultrasound transducer assembly comprises an orientation scale that permits visual determination of the orientation of the ultrasound transducer relative to the housing.

In some example implementations of the ultrasound device, the housing is configured to facilitate variation of the lateral position of the ultrasound transducer assembly relative to the housing, and wherein at least one of the housing and the ultrasound transducer assembly comprises a lateral position scale that permits visual determination of the lateral position of the ultrasound transducer relative to the housing.

In some example implementations, the ultrasound device further comprises two or more marking apertures that permit marking of the skin surface after securing the housing relative to the skin surface, thereby facilitating re-alignment of the ultrasound device after detachment from the skin surface and re-attachment of the housing to the skin surface.

In some example implementations, the ultrasound device further comprises at least one motor operably coupled to the ultrasound transducer assembly; and control circuitry configured to control actuation of the at least one motor for controlling of at least one of the orientation and the lateral position of the ultrasound transducer assembly relative to the housing.

The ultrasound device may further comprise an encoding mechanism for encoding at least one of the orientation and the lateral position of the ultrasound transducer assembly relative to the housing, wherein the control circuitry is configured to process a signal from an encoding sensor associated with the encoding mechanism to determine at least one of the orientation and the lateral position of the ultrasound transducer assembly relative to the housing.

The ultrasound device may further comprise a reference sensor for detecting at least one of a reference angle and a reference lateral position of the ultrasound transducer assembly relative to the housing, wherein the control circuitry is configured to process a signal from the reference sensor to determine when the ultrasound transducer assembly resides at the reference angle or the reference lateral position.

In some example implementations of the ultrasound device, the ultrasound transducer assembly and the housing are configured such that the ultrasound transducer assembly is moveable, relative to the housing, for varying the orientation and the lateral position of the ultrasound transducer assembly.

In some example implementations of the ultrasound device, the ultrasound transducer is a single element ultrasound transducer.

In some example implementations of the ultrasound device, the ultrasound transducer comprises a plurality of ultrasound transducer elements.

In some example implementations, the ultrasound device further comprises a flexible substrate that is attachable to the skin surface for indirectly supporting the housing relative to the skin surface.

In another aspect, there is provided an ultrasound device comprising:
a housing configured for attachment to a skin surface;
an ultrasound transducer supported relative to the housing such that the ultrasound transducer is moveable, relative to the housing, for varying at least one of:
   an orientation of the ultrasound transducer relative to the housing; and
   a lateral position of the ultrasound transducer relative to the housing; and
a locking means for releasably locking at least one of the orientation and the lateral position of the ultrasound transducer relative to the housing.

In another aspect, there is provided an ultrasound device comprising:
a housing configured for attachment to a skin surface;
an ultrasound transducer supported relative to the housing such that the ultrasound transducer is moveable, relative to the housing, for varying at least one of:
   an orientation of the ultrasound transducer relative to the housing; and
   a lateral position of the ultrasound transducer relative to the housing; and
a scale that permits visual determination of at least one of the orientation and lateral position of the ultrasound transducer relative to the housing.

In another aspect, there is provided an ultrasound device comprising:
a housing configured for attachment to a skin surface;
an ultrasound transducer assembly supported by the housing, the ultrasound transducer assembly comprising:
   an ultrasound transducer; and
   a coupling component for coupling ultrasound energy between the ultrasound transducer and the skin surface, wherein the coupling component is in acoustic communication with the ultrasound transducer, and wherein a distal portion of the coupling component is configured to contact and apply pressure to the skin surface when housing is secured to the skin surface; and
wherein the ultrasound transducer assembly and the housing are configured such that the ultrasound transducer assembly is moveable, relative to the housing, for an orientation of the ultrasound transducer assembly relative to the housing;
the ultrasound transducer assembly being supported, relative to the housing, such that a constant pressure is maintained between the distal portion of the coupling component and the skin surface when the ultrasound transducer assembly is moved relative to the housing.

In another aspect, there is provided an ultrasound device comprising:
a housing configured for attachment to a skin surface;
an ultrasound transducer assembly supported by the housing, the ultrasound transducer assembly comprising:
   an ultrasound transducer; and
   a coupling component for coupling ultrasound energy between the ultrasound transducer and the skin surface, wherein the coupling component is in acoustic communication with the ultrasound transducer, and wherein a distal portion of the coupling component is configured to contact and apply pressure to the skin surface when housing is secured to the skin surface; and
wherein the ultrasound transducer assembly and the housing are configured such that the ultrasound transducer assembly is moveable, relative to the housing, for varying a lateral position of the ultrasound transducer assembly relative to the housing;
the ultrasound transducer assembly being supported, relative to the housing, such that a constant pressure is maintained between the distal portion of the coupling component and the skin surface when the ultrasound transducer assembly is moved relative to the housing.

In another aspect, there is provided an ultrasound device comprising:
a housing configured for attachment to a skin surface;
an ultrasound transducer supported relative to the housing such that the ultrasound transducer is moveable, relative to the housing, for varying at least one of:
   an orientation of the ultrasound transducer relative to the housing; and
   a lateral position of the ultrasound transducer relative to the housing; and
wherein the housing is configured to generate a frictional force as a result of contact with the ultrasound transducer assembly, wherein the frictional force is sufficiently low to permit manual actuation thereof and sufficiently high to maintain the ultrasound transducer assembly in a fixed orientation in the absence of an external applied force.

In another aspect, there is provided a method of performing Doppler ultrasound, the method comprising:
securing the ultrasound device according to claim 1 to the skin surface of a subject;
while actuating the ultrasound device to vary at least one of the orientation of the ultrasound transducer assembly relative to the housing and the lateral position of the ultrasound transducer assembly relative to the housing:
   detecting Doppler ultrasound signals from the ultrasound transducer; and
   employing the Doppler ultrasound signals to determine at least one of a suitable orientation and a suitable lateral position of the ultrasound transducer assembly that is associated with detection of blood flow within a blood vessel.

In some example implementations of the method, the ultrasound device further comprises a locking means for releasably locking at least one of the suitable orientation and the suitable lateral position of the ultrasound transducer relative to the housing, the method further comprising: after determining at least one of the suitable orientation and the suitable lateral position that is associated with detection of blood flow within the blood vessel, actuating the locking means.

In some example implementations of the method, the ultrasound transducer assembly and the housing are configured such that the ultrasound transducer assembly is moveable, relative to the housing, for varying both the orientation and lateral position of the ultrasound transducer assembly relative to the housing, and wherein the locking means is configured for releasably locking both the orientation and the lateral position of the ultrasound transducer assembly relative to the frame, the method further comprising: after varying both the orientation and lateral position of the ultrasound transducer assembly to determine the suitable orientation and the suitable lateral position of the ultrasound transducer that is associated with detection of blood flow within the blood vessel, actuating the locking means to lock both the orientation and the lateral position of the ultrasound transducer assembly.

In some example implementations of the method, the ultrasound device further comprises a scale that permits visual determination of at least one of the orientation and lateral position of the ultrasound transducer relative to the housing, the method further comprising: employing the scale to record the at least one of the orientation and lateral position of the ultrasound transducer that is associated with detection of blood flow within the blood vessel.

In some example implementations of the method, the ultrasound transducer comprises a one-dimensional array of ultrasound elements disposed along a longitudinal axis, and wherein the ultrasound transducer assembly and the housing are configured such that the ultrasound transducer assembly is moveable, relative to the housing, for varying both the orientation and lateral position of the ultrasound transducer assembly relative to the housing, the method further comprising: employing the Doppler ultrasound signals to determine the suitable orientation and the suitable lateral position of the ultrasound transducer assembly such that the longitudinal axis is substantially perpendicular to an axis of the blood vessel.

In some example implementations of the method, the housing is indirectly secured to the skin surface via an intermediate flexible substrate adhered to the skin surface.

In another aspect, there is provided a system for performing Doppler ultrasound, the system comprising:
a linear array of ultrasound transducer elements;
a support structure supporting the linear array of ultrasound transducer elements, the support structure comprising a coupling component having a flexible distal surface configured to contact and conform to a skin surface of a subject, the linear array of ultrasound transducer elements being supported such that when the distal surface is contacted with and conforms to the skin surface of the subject, a respective beam axis of each ultrasound transducer element extends through the coupling component and through the skin surface, at an oblique angle relative to the skin surface, and such that respective focal regions associated with the ultrasound transducer elements reside beneath the skin surface and are absent of spatial overlap with each other; and
control and processing circuitry connectable to the linear array of ultrasound transducer elements, the control and processing circuitry comprising at least one processor and memory, the memory comprising instructions executable by the at least one processor, when the control and processing circuitry is connected to the linear array of ultrasound transducer elements, for performing operations comprising:
   controlling each ultrasound transducer element to transmit a respective ultrasound beam therefrom;
   receiving, from each ultrasound transducer element, a respective receive ultrasound signal;
   processing each receive ultrasound signal to generate a respective Doppler signal, such that each Doppler signal is associated with a different ultrasound array element;
   processing the Doppler signals to identify one or more Doppler ultrasound signals that satisfy pre-selected criterion; and
   employing the Doppler signals that satisfy the pre-selected criterion to generate a hemodynamic measure.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 shows the major arteries that comprise the posterior circulation of the foot.

FIG. 2 shows the major arteries that comprise the anterior circulation of the foot.

FIG. 3 shows the pedal arch, which is the connection between the anterior and posterior circulation of the foot.

FIG. 4 shows a typical ultrasound image of the foot (top of image) with blood flow velocity waveform (bottom of image) and the measuring technique used to obtain PAT.

FIG. 5 shows four classifications of ischemia and how they correlate to PAT, clinical symptoms, and Ankle Brachial Index (ABI).

FIG. 6A shows the top view (left side of figure) and side view (right side of picture) of an example embodiment in which an ultrasound sensor is placed on the top of the foot.

FIG. 6B shows a top view (right side) and cross section view (left side) of the example embodiment shown in FIG. 6A.

FIG. 6C shows a system diagram of an example embodiment of the ultrasonic system.

FIG. 7A shows an example ultrasonic transducer patch comprising a housing, transducers, and a conformable coupling wherein the transducers are generally parallel to the bottom surface of the housing and the coupling creates an angle between the vessel and the transducers in order to optimize the Doppler sensitivity and accuracy.

FIG. 7B shows an example ultrasonic transducer patch comprising a housing, transducers, and a conformable coupling wherein the transducers are generally angled relative to the bottom surface of the housing, creating an angle between the vessel and the transducers in order to optimize the Doppler sensitivity and accuracy.

FIG. 8 shows the top view (left side of figure) and side view (right side of picture) of an example embodiment in which an ultrasound sensor is placed on the top of the foot. The sensor consists of multiple ultrasound transducers connected with a flexible substrate.

FIG. 9 shows the top view (left side of figure) and side view (right side of picture) of an example embodiment in which a radiopaque grid is first affixed to the foot. A single ultrasound sensor is then placed at the optimal grid coordinates determined from fluoroscopic imaging.

FIG. 10A shows an example embodiment in which a transducer is housed within a ball which can rotate within a socket-shaped housing. The housing is connected to a flexible, adhesive strip.

FIG. 10B shows a cross sectional view of the example embodiment shown in FIG. 10A.

FIG. 10C shows the ball in FIGS. 10A and 10B rotated relative to the socket-shaped housing.

FIG. 10D shows a cross sectional view of the example embodiment shown in FIG. 10C.

FIG. 11 shows a cross sectional view and top view of an example embodiment which comprises a flexible adhesive strip, one or more ultrasound transducers situated within a slider, and a housing. The slider can be translated along a track in the housing to adjust the position of the transducers after placement with adhesive strip.

FIG. 12 shows a cross sectional view of an example configuration wherein the geometry causes the ultrasonic transducer to be angled relative to the velocity of the vessel blood flow.

FIG. 13 shows a cross sectional view of another example configuration wherein the geometry causes the ultrasonic transducer to be angled relative to the velocity of the vessel blood flow.

FIG. 14 shows orthogonal cross-sectional views of another example configuration wherein the skin contacting portion of the housing is semi-cylindrically shaped.

FIG. 15 shows orthogonal cross-sectional views of another example configuration wherein the skin contacting portion of the housing is semi-spherically shaped and located between two bones.

FIG. 16 shows an anatomic area between the first and second metatarsals known as the Cuneiform window.

FIG. 17 shows an example embodiment wherein multiple ultrasonic transducers are positioned substantially parallel to each other and the vessel, but pulsed in a manner so as to steer the beam to an optimal Doppler angle.

FIG. 18 shows an example embodiment in which the housing includes locating holes to assist in marking of the patient and placement of the device.

FIG. 19 shows an example embodiment wherein a radiotransparent retainer is held in position on the patient's foot with an adhesive coupling component. An ultrasonic transducer can be inserted and removed from the retainer as needed to allow for clear fluoroscopic imaging.

FIG. 20 shows a schematic diagram of an example embodiment of the ultrasound system.

FIG. 21 shows an ultrasound patch with multiple transducer elements affixed to the patient's foot.

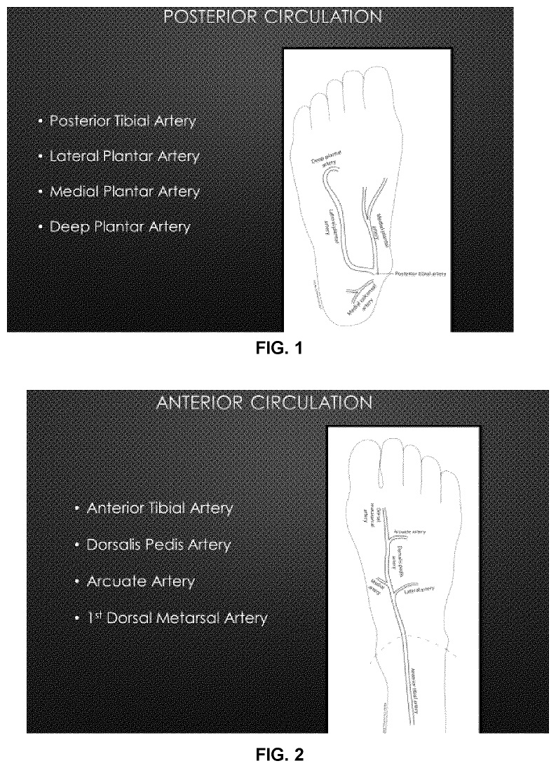

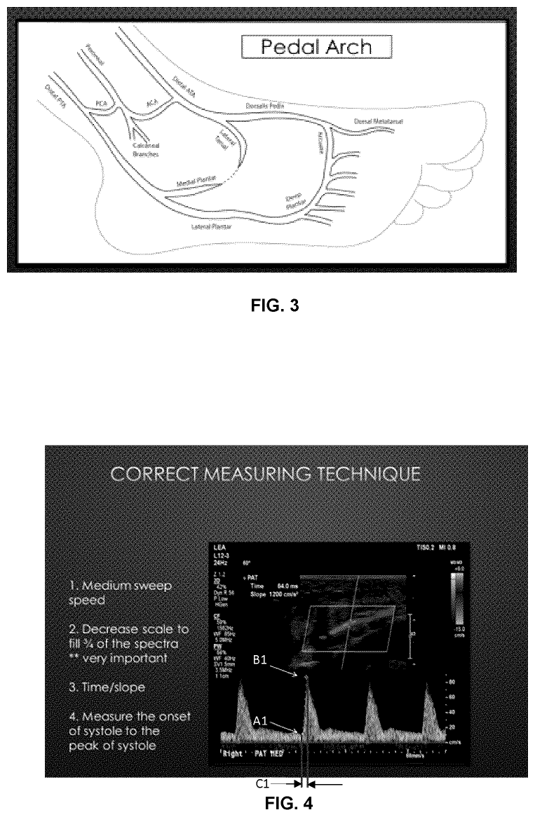

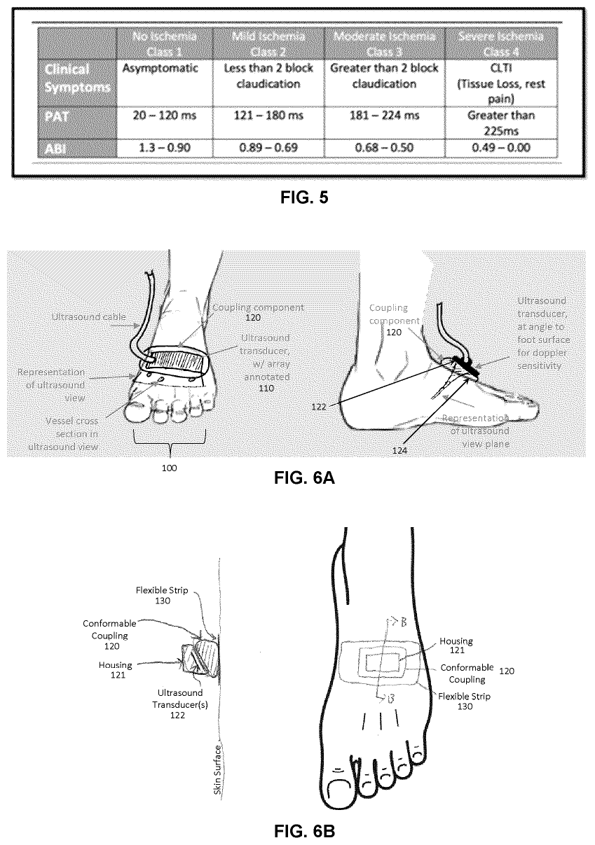

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Despite the aforementioned initial success of PAT in demonstrating clinical utility in the diagnosis and management of PAD and CLTI, there are a number of challenges to implementing this technology. Firstly, obtaining hemodynamic measurements requires a skilled and trained ultrasound operator who is trained not only in performing vascular imaging, but is also trained in the anatomy of the foot. There is a lack of skilled and properly trained professionals that can properly perform these studies. Secondly, many physicians do not have access to ultrasound professionals in the operating room or catheter lab. This in turn requires the physicians themselves to perform the ultrasound hemodynamic measurements for PAT, which can be cumbersome and time consuming, thus rendering it challenging for them to obtain these measurements themselves during a procedure. Thirdly, standard ultrasound equipment can be bulky, expensive, and have numerous features that require extensive training, limiting their use and clinical utility in the operating room or catheter lab.

In view of these challenges, there is therefore a clear need for an ultrasound device that is capable of measuring PAT and/or other hemodynamic measures, and which requires minimal training and can be used in a wide variety of clinical settings, such as the catheter lab, operating room and/or other remote settings such as in the physician's office or the patient's home.

Various example embodiments of the present disclosure solve the aforementioned problems by providing an ultrasound device that facilitates the positioning and orientation of an ultrasound beam relative to the vasculature of the patient. As will be explained in detail below, the present example embodiments may be useful in facilitating the measurement of PAT and other hemodynamic measures by untrained operators in a wide variety of clinical settings.

Some example embodiments of the present disclosure provide an ultrasound system that includes an ultrasound device in the form of a patch having transducers supported thereon, where the patch is removably attached to the foot. Such systems may be employed to measure blood flow through the major and/or minor vessels of the pedal arch. Such systems may also be employed to detect blood velocity waveforms and calculate pedal acceleration time, among other physiologic parameters.

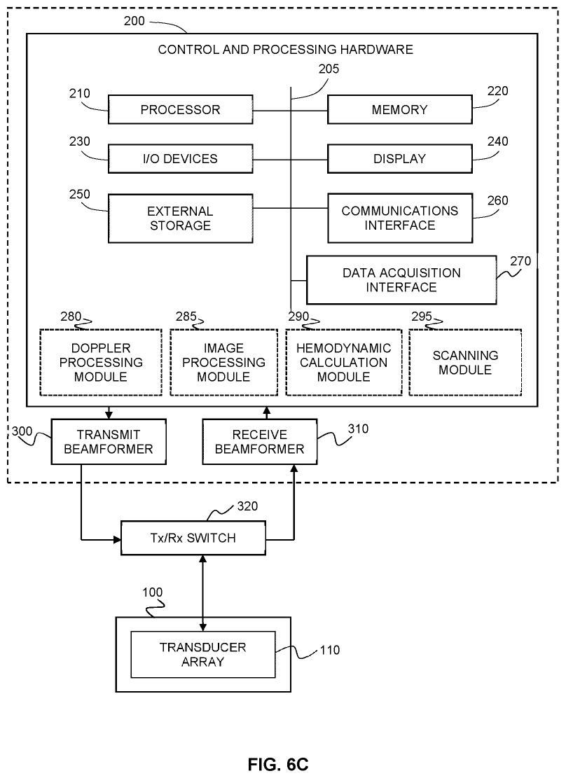

In one example embodiment, shown in FIG. 6A, an ultrasound device 100 includes a rigid ultrasound transducer array 110 that is coupled to the top of the foot with a conformable coupling material 120 and operable as a phased-array ultrasound transducer. As shown in the figure, the ultrasound transducer array 110 may be rigidly supported in a curved configuration. The top surface 122 of the coupling material 120 can be configured to be temporarily or permanently attached to the transducer array 110. For example, with adhesive, thermal bonding, or mechanical interlocks. The opposing bottom surface 124 of the coupling material 120 may be removably attachable to the foot, for example, with an adhesive.

FIG. 6B illustrates an example implementation in which a flexible, adhesive strip 130 is attached to the coupling material 120 to secure the device to the patient. As shown in the figure, the coupling material 120 conforms to the curvature of the foot and facilitates the transmission of ultrasound waves from the device to the foot. A housing 121 may be made of a rigid material such as polycarbonate plastic and holds one or more ultrasonic transducers 122. The housing 121 may be permanently or removably secured to the coupling material 120, for example with adhesive, thermal bond, or mechanical interlock.

In some example implementations, the coupling material 120 can be made of silicone, hydrogel, a gel-filled bag, or other compliant materials.

FIG. 6C shows an example system for performing ultrasound measurements using an ultrasound transducer array 110 that is operated in a phased-array mode. The example system includes ultrasound device 110 that includes ultrasound transducer array 110, a transmit beamformer 300 with pulser-receiver circuitry 320, a receive beamformer 310 and control and processing hardware 200 (e.g. a controller, computer, or other computing system).

Control and processing hardware 200 is employed to control transmit beamformer 300 and receive beamformer 310, and for processing the beamformed receive signals. As shown in FIG. 6C, in one embodiment, control and processing hardware 200 may include a processor 210, a memory 220, a system bus 205, one or more input/output devices 230, and a plurality of optional additional devices such as communications interface 260, display 240, external storage 250, and data acquisition interface 270.

The present example methods involving the control of the ultrasound transducer array 110 for performing hemodynamic measurements (e.g. the detection of PAT) can be implemented via processor 210 and/or memory 220. As shown in FIG. 6C, the control of the ultrasound transducer array 110 may be implemented by control and processing hardware 200, via executable instructions represented as image processing module 290. The control and processing hardware 200 may include and execute scan conversion software (e.g. real-time scan conversion software).

In some example implementations, the transducer array 110 is controlled to obtain one or more Doppler (flow velocity) waveforms, as indicated by Doppler processing module 280. In some example embodiments, the transducer array may be controlled to scan the ultrasound beam and generate an ultrasound image, for example, as controlled via image processing module 285. Each Doppler waveform may correspond to a different location, such as a location within an image acquired by the ultrasound transducer array 110. The ultrasound image may be a Doppler ultrasound image.

In some example implementations, the control and processing hardware 200 may be employed to process a given Doppler waveform in order to calculate one or more hemodynamic measures, such as the PAT, as schematically shown by hemodynamic calculation module 290. For example, a Doppler waveform may be processed to calculate the PAT by calculating the time from the beginning of systole to the peak of systole. Velocity waveforms may also be captured.

It will be understood that the PAT is but one example of a hemodynamic measure that can be determined via the processing of one or more Doppler waveforms. Non-limiting examples of additional or alternative hemodynamic measures include flow volume, peak velocity, beats per minute, and velocity slope from start to end of systole.

In some example implementations, the ultrasound beam transmitted by the ultrasound transducer array 110 may be scanned in order to identify one or more regions associated with a sufficiently high Doppler signal, such as a location corresponding to a vessel of interest for performing hemodynamic measurements. For example, the transmit beamformer 300 can be controlled to scan the ultrasound beam across a 1D or 2D angular range (2D if the transducer array is a 2D transducer array) and the Doppler signals may be processed to determine an angle that corresponds to maximal signal, as schematically shown by scanning module 295. In some example implementations, a preferred angle may be determined by processing the collected Doppler signals according to a machine learning algorithm, such as a neural network, that was trained with Doppler signals having a desired shape and/or signal-to-noise ratio.

Alternatively, input from an operator can be employed to control steering of the ultrasound beam, and/or to indicate the angle at which a Doppler signal is deemed to correspond to a region of interest, such as a vessel of interest. For example, the device can output both Doppler audio and a visual Doppler waveform to the operator. The operator can use this feedback to steer the beam so as to get the best signal. As the beam is steered closer to the vessel, the audio volume will increase, the Doppler sound will become more of a well-defined pulsatile flow sound, the velocity waveform will become more defined, and systolic peaks will increase. As the beam is steered further from the vessel, the audio volume will decrease, the Doppler sound will become less of a well-defined pulsatile flow sound, the velocity waveform will become less defined, and systolic peaks will decrease.

The functionalities described herein can be partially implemented via hardware logic in processor 210 and partially using the instructions stored in memory 220. Some embodiments may be implemented using processor 210 without additional instructions stored in memory 220. Some embodiments are implemented using the instructions stored in memory 220 for execution by one or more general purpose microprocessors. In some example embodiments, customized processors, such as application specific integrated circuits (ASIC) or field programmable gate array (FPGA), may be employed. Thus, the disclosure is not limited to a specific configuration of hardware and/or software.

It is to be understood that the example system shown in FIG. 6C is not intended to be limited to the components that may be employed in a given implementation. For example, the system may include one or more additional processors. Furthermore, one or more components of control and processing hardware 200 may be provided as an external component that is interfaced to a processing device. For example, as shown in the figure, any one or more of transmit beamformer 300 and receive beamformer 310 may be included as a component of control and processing hardware 200 (as shown within the dashed line), or may be provided as one or more external devices.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed herein can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

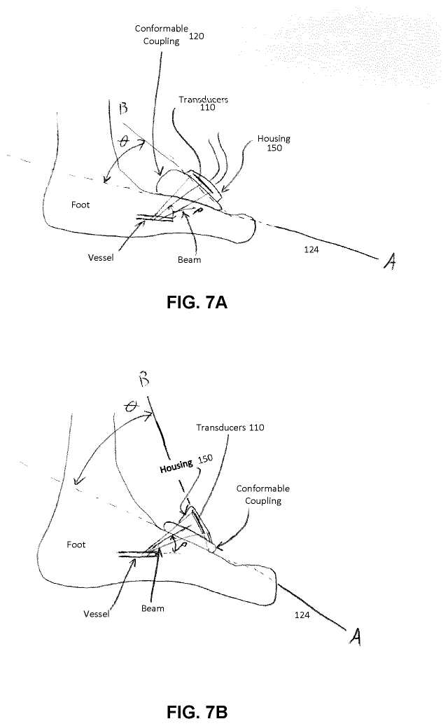

In order to perform a Doppler measurement, it may be beneficial for the ultrasound beam to be directed at an angle relative to the vessel flow direction, such an angle p between the ultrasound beam axis and a local axis of the vessel of interest is less than 75 degrees, as shown in FIGS. 7A and 7B. This can be achieved, for example, by shaping the conformable coupling material 120 (as shown in FIG. 7A) such that the ultrasound transducer array 110 (i.e. an axis intersecting the elements of the array) is angled relative bottom surface 124 at an angle θ of greater than 15°. Alternatively, as shown in FIG. 7B, a housing 150 supporting the ultrasound transducer array 110 may be provided such that the ultrasound transducer array 110 (i.e. an axis intersecting the elements of the array) is angled relative bottom surface 124 at an angle θ of greater than 15°.

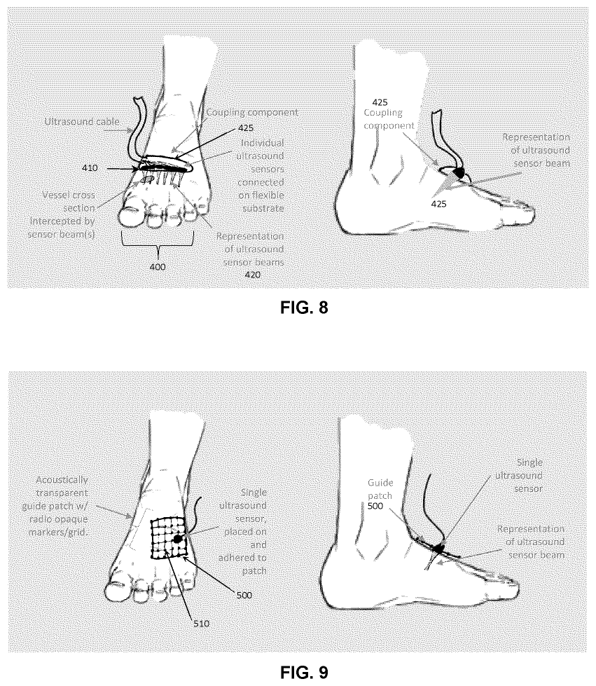

FIG. 8 illustrates an alternative example ultrasound device 400 in which the ultrasound transducer array is a linear array 410 that is operated to generate a plurality of ultrasound beams 420, as opposed to a single phased-array ultrasound beam. Each ultrasound beam 420 may be generated by a subset of one or more transducer elements. The ultrasound beams 420 may be generated such that two or more ultrasound beams are generated temporally in parallel or temporally in series. For example, the linear array may be operated to generate a single ultrasound beam 420 at any one given time, using a single transducer element or a set of adjacent transducer elements.

The linear array 410 of ultrasound transducer elements is supported by a substrate that may be rigid or flexible and may be removably affixed to the patient with adhesive. Alternatively, the substrate may be affixed to a flexible strip (not shown), which in turn may be removably affixed to the patient with adhesive. In example implementations in which the device is flexible, the ultrasound array may conform to the anatomical curvature of the foot.

In some example embodiments, the support structure includes a coupling component 425 having a flexible distal surface that is configured to contact and conform to the skin surface of the subject. The linear array of ultrasound transducer elements 410 may be supported such that when the distal surface is contacted with and conforms to the skin surface of the subject, a respective beam axis of each ultrasound transducer element extends through the coupling component 425 and through the skin surface, at an oblique angle relative to the skin surface, as shown in FIG. 8. Furthermore, the ultrasound array elements 410 may be supported such their focal regions reside beneath the skin surface and are absent of spatial overlap with each other.

In some example implementations, the ultrasound signals received by the linear array 410 may be processed in order to identify one or more Doppler signals that satisfy pre-selected criterion, and to employ such Doppler signals to generate a hemodynamic measure. For example, the criterion may be selected to identify Doppler signals that have a sufficiently high signal or a Doppler signal with a sufficiently high signal-to-noise ratio. Such criterion can be employed, for example, to identify and select Doppler signals that correspond to a vessel of interest for performing hemodynamic measurements. For example, referring to FIG. 6C, the transmit beamformer 300 can be controlled to sequentially generate a series of ultrasound beams 410 and detect respective Doppler signals, and the Doppler signals may be processed to determine a configuration (e.g. a selected beam or selected set of one or more transducer elements) that corresponds to maximal signal, as schematically shown by scanning module 295. In some example implementations, a preferred beam may be determined by processing the collected Doppler signals according to a machine learning algorithm, such as a neural network, that was trained with Doppler signals having a desired shape and/or signal-to-noise ratio.

Alternatively, input from an operator can be employed to determine a desired beam configuration, and/or to indicate the beam and/or set of one or more ultrasound transducer elements for which a Doppler signal is deemed to correspond to a region of interest, such as a vessel of interest. For example, the operator may be able to turn individual transducer elements on and off while observing the waveform and listening to audio Doppler to determine which transducer elements provide the optimal signals.

The ultrasound array of ultrasound transducers can be operated according to a wide variety of modalities that facilitate the detection of a Doppler signal. For example, the ultrasound array can be operated according to one or more modes selected from continuous wave (CW) mode (e.g. CW Doppler, optionally steered if implemented in a phased-array transducer configuration), pulse wave mode (e.g. pulsed wave Doppler), or power wave Doppler ultrasound (ultrasound angiography) mode.

FIG. 9 illustrates an example device and method of vessel alignment. As shown in the figure, a flexible substrate 500 has a plurality of reference markings and is affixed to the foot of the patient. The flexible substrate 500 is radio-transmissive such that a fluoroscopy of the foot may be acquired while the flexible substrate resides on the foot. Non-limiting examples of suitable materials for the flexible substrate include mylar, silicone, and urethane film. The reference markings may take any suitable form, such as an array of dots, an array of numbers, and a set of grid lines 510, with the latter shown in FIG. 9. The reference markings 510 are preferably radiopaque and can be seen when viewed under x-ray. In some non-limiting example implementations, the reference markings may be formed by a radiopaque ink or by metal wire. In some example implementations, one or more geometrical reference markings, such as a line or axis, may be labeled with a symbol, such as a number and/or letter.

In one example embodiment, the bottom surface 520 of the flexible substrate is affixed to the patient with adhesive. When viewed under fluoroscopy, an operator can employ the reference markings to identify a location or a set of locations that is aligned with a target vessel. The operator can use this information to center and secure an ultrasound transducer to the grid at the desired location with adhesive or a flexible adhesive strip. The transducer may be an ultrasound transducer array (e.g. a phased array or linear array) or a single ultrasound transducer (as shown in FIG. 9).

As described in many example embodiments of the present disclosure, an ultrasound device may be provided that includes a housing having a shape (e.g. an outer surface feature or flange) configured for or suitable to be secured to a skin surface. The housing supports an ultrasound transducer and a coupling component for coupling ultrasound energy beneath the skin surface and from the skin surface to the ultrasound transducer. In some example implementations, a flexible substrate may be provided that is configured for attachment to a skin surface (e.g. via an adhesive), where the flexible substrate supports (optionally removably supports) the housing, such that the housing is indirectly secured to the skin surface.

In some example embodiments, the orientation of the ultrasound transducer and/or the lateral position of the ultrasound transducer may be movable relative to the housing. The housing and/or the ultrasound transducer assembly may include a feature or mechanism that enables the variation of the orientation of the ultrasound transducer assembly relative to the housing and/or the variation of the lateral position of the ultrasound transducer assembly relative to the housing. The phrase “lateral”, when referring to varying the lateral position or performing lateral translation, is intended to mean a change in the position of the ultrasound transducer (or the ultrasound transducer assembly) in a direction that is parallel to the substrate (parallel to the skin surface, or a local tangent associated with the skin surface).

In some example embodiments, the ultrasound device may be actuated to vary the orientation and/or lateral position of a transducer assembly that includes both the ultrasound transducer and a coupling component (with the ultrasound transducer optionally embedded in, or partially surrounded, by the coupling component) where the coupling component is in acoustic communication with the ultrasound transducer. In some example implementations, the transducer assembly may be supported, relative to the housing, such that a distal portion of the coupling component contacts and applies pressure to the skin surface when the housing is attached to the skin surface (i.e. the coupling component locally depresses the skin, relative to the outer skin surface surrounding the housing). This contact may optionally be facilitated via a coupling gel that is disposed between the coupling component and the skin surface.

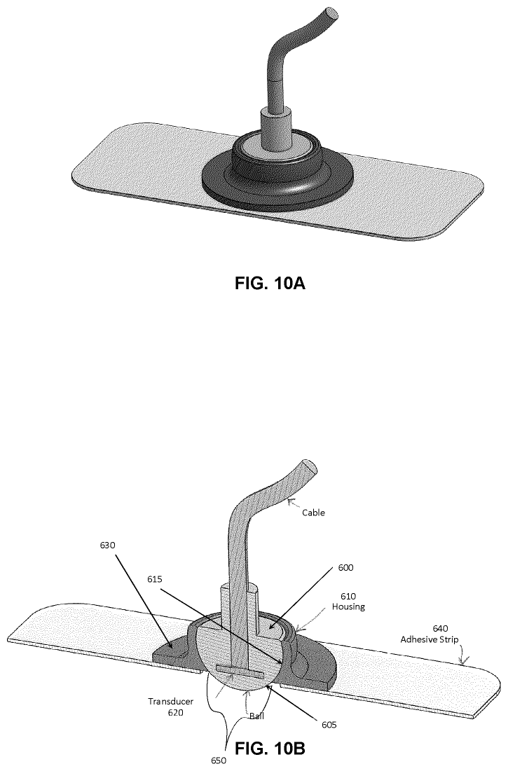

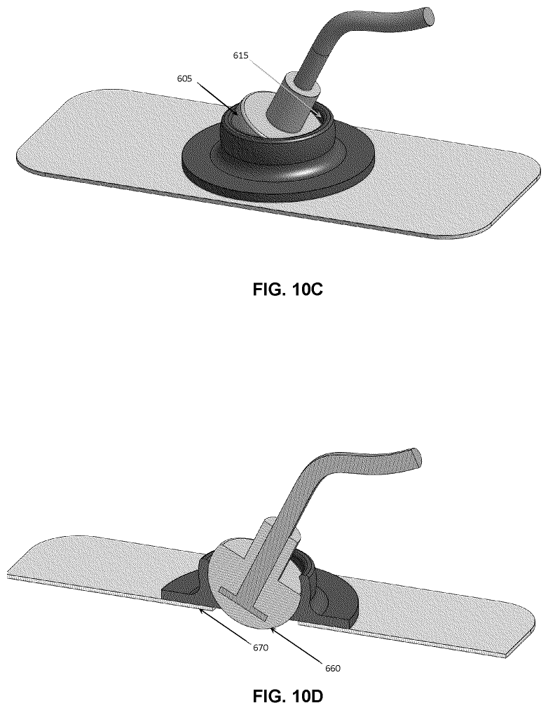

An example of such a configuration is illustrated in FIGS. 10A-10D, which illustrate an example embodiment that facilitates steering and/or scanning of an ultrasound beam while maintaining contact of the distal region of the coupling component with the skin surface. An ultrasound transducer 620 (e.g. a single ultrasound transducer element or an ultrasound transducer array) is supported within a transducer assembly 600 that forms a pivotable structure. The ultrasound transducer is surrounded by (embedded within) and acoustically coupled to an outer coupling component 625 . . . . As shown in the figure, at least a distal portion of the ultrasound transducer assembly 600 is spherical in shape and resides within a socket defined by the inner cavity of the housing 610, thereby forming a ball and socket joint. In the present example embodiment, the housing 610 (socket) has a distal aperture through which the ultrasound transducer assembly 600 projects, such that a distal portion of the coupling component 625 contacts and applies pressure to the skin surface when the housing 610 is attached to the skin surface. The ultrasound transducer assembly (pivot structure) 600 has an outer surface 605 that maintains contact with an inner surface 615 of the housing 610 as the ultrasound transducer assembly 600 is pivoted relative to the housing 610.

In some example implementations, friction between the housing 610 and the ultrasound transducer assembly 600 is sufficiently low to permit the ultrasound transducer assembly 600 to be pivoted by a user, but sufficiently high to maintain the ultrasound transducer assembly 600 in a desired orientation in the absence of an external applied force. In one example implementation, an operator can manually steer the ultrasound transducer assembly (and thus the one or more ultrasound transducer elements) relative to the patient, without translation of the pivot structure relative to the patient, according to one or two rotational degrees of freedom after securing the housing 610 relative to the patient. As described below, the present example embodiment may also be implemented in an automated configuration, the change in angle being robotically actuated, e.g. by a motor and/or robotic arm.

As shown in FIG. 10B, at least a portion of the ultrasound transducer assembly 600 may have a spherical outer surface 605, and at least a portion of the housing 610 may have in inner spherical surface 615, where the spherical inner surface 615 of the housing 610 is configured to contact the outer surface 605 of the ultrasound transducer assembly while the ultrasound transducer assembly 600 is pivoted relative to the housing 610, thus assisting in retaining the ultrasound transducer assembly 600 in a secured position while it is pivoted. The ultrasound transducer 620 may be embedded within the pivot structure 600, as shown in FIG. 10B. The housing 610 and the coupling component 625 may be formed from a rigid or semi-rigid material.

The housing 610 has a surface portion that is configured to facilitate attachment of the housing 610 to the patient's anatomy, either directly or indirectly via attachment to a flexible strip or patch. For example, the housing 610 has an outer flange portion 630 that may be directly secured to the patient's foot. Alternatively, as shown in the figures, the outer flange 630 may be secured to a flexible strip or patch 640 that is removably attachable to the patient.

In the present example embodiment shown in FIG. 10B, an aperture or recess 650 may be formed in the housing, such that ultrasound energy generated by the ultrasound transducer can enter the patient in the absence of propagation through the housing, optionally through an additional coupling medium disposed between the pivot structure and the patient (not shown).

According, in some example embodiments, the pivot structure 600 may be secured within the housing 610 such that a distal portion 660 of the pivot structure protrudes through an aperture defined within the housing 610. The pivot structure 600 may be secured within the housing 610 such that the distal portion 660 of the pivot structure extends (protrudes) below a lower contact surface 670 of the housing, such that when the lower contact surface 670 of the housing 610 is secured to the patient (either directly or indirectly), the distal portion 660 of the pivot structure 600 applies a compressive force to the patient, thereby facilitating the coupling of ultrasound energy from the pivot structure 600 to the patient. Indeed, the spherical shape of the outer surface ensures that a constant pressure is maintained between the coupling component and the skin surface during rotation of the ultrasound transducer assembly. This aspect can be beneficial in many applications, especially those involving Doppler ultrasound detection in peripheral vasculature, such as, but not limited to, pedal ultrasound Doppler detection and imaging. The protrusion of the distal region of the pivot structure 600 may also be employed to assist in positioning the device such that the distal region resides between two anatomic bony structures, such as the first and second metatarsals.

In other example embodiments, the ultrasound assembly 600 and the housing 610 may be configured such that ultrasound energy generated by the ultrasound transducer 620 propagates through an acoustically transmissive distal coupling portion of the housing 610 (not shown) prior to entering the patient. The distal coupling portion of the housing may contact and apply pressure to the skin surface when the housing 610 is attached to the skin surface, with the housing secured thereon, while providing acoustic coupling between the skin surface and the moveable coupling component of the ultrasound transducer assembly 600.

The distal coupling portion of the housing may have a spherical inner surface that matches the outer spherical surface of the ultrasound transducer assembly, and acoustic coupling between the distal coupling portion of the housing and the ultrasound transducer assembly 600 may be enhanced by a thin layer of acoustic coupling gel or fluid disposed between the ultrasound transducer assembly 600 and the inner surface of the distal coupling portion of the housing 610. Accordingly, in the present example implementation, a constant spacing may be maintained between the ultrasound transducer assembly 600 and the distal coupling region of the housing during rotation of the ultrasound transducer assembly, thereby ensuring that a constant pressure is applied to the skin surface during rotational of the ultrasound transducer assembly.

While FIGS. 10A-10D illustrate an example embodiment involving the use of a ball and socket joint to facilitate rotational motion of the transducer assembly, it will be understood that this example embodiment is not intended to be limiting, and that a wide range of configurations and mechanisms may be employed to facilitate rotation of the ultrasound transducer assembly relative to the housing. For example, in some example implementations, a pivot mechanism (e.g. a pivot pin, or set of pivot pins, and associated divots or recesses, or hinge) may be employed to facilitate rotation about a rotation axis. In such an example implementation, a distal surface of the coupling component may have a circular shape within a plane perpendicular to the rotation axis, such that the constant pressure is maintained between the coupling component and the skin surface (or a distal coupling portion of the housing) during rotation of the ultrasound transducer assembly about the rotation axis.

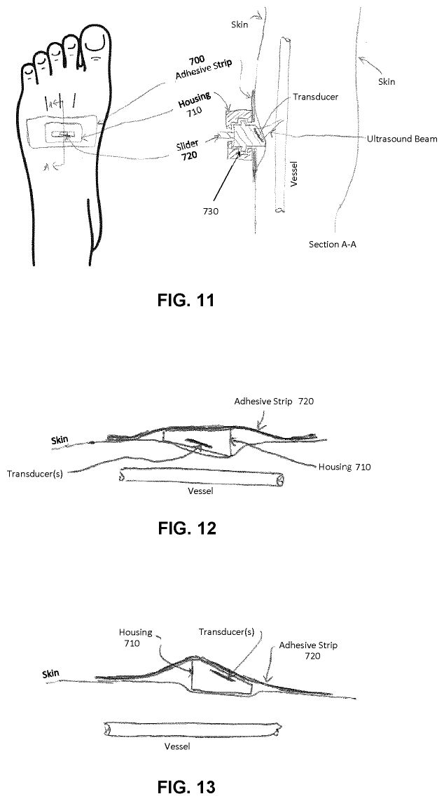

Referring now to FIG. 11, an example embodiment is illustrated in which an ultrasound transducer assembly is supported by a housing 710 such that the ultrasound transducer assembly is translatable, in a lateral direction, relative to the housing 710. An adhesive strip 700 is optionally employed to secure the housing 710 relative to a patient. An ultrasonic transducer (e.g. a single ultrasound transducer element or a transducer array) is housed within a moveable ultrasound transducer assembly 720 (e.g. a “slider”) that is supported by the housing 710 and movable relative to the housing 710.

It will be understood that a wide variety of mechanisms may be employed to facilitate lateral translation of the ultrasound transducer assembly 720. For example, the housing 710 may include a track 730 and the movable ultrasound transducer assembly 720 may be moveable relative to a track, thereby enabling a user to translate the ultrasound transducer along the track. In some example implementations, the operator may monitor a Doppler signal (or other ultrasound hemodynamic signal) in order to achieve a desired signal level that is sufficient for measuring one or more hemodynamic measures.

As shown in the figure, the housing 710 may be secured to the skin surface such that a distal portion of the ultrasound transducer assembly that is configured to contact the patient contacts the patient skin surface at an angle in order to facilitate the detection of a Doppler signal, and optionally locally applies pressure by depressing the skin surface. Moreover, can be understood from the figure, when such pressure is applied, the lateral translation of the ultrasound transducer assembly 720 results in the maintaining of a constant pressure between the distal portion of the ultrasound transducer assembly (i.e. a distal coupling component) and the skin surface.

It is noted that when implementing either of the example embodiments shown in FIGS. 10A-D and FIG. 11, an operator can initially position the device with the application of the adhesive strip, and then employ movement of the pivotable or movable component to fine-tune the signal acquisition or adjust as necessary after adhesive application.

While the figures and preceding description refer to example implementations in which the ultrasound device is configured to facilitate either rotation or lateral translation of the ultrasound transducer assembly relative to the housing, it will be understood that other example implementations may combine such features, such that the ultrasound device is configured to facilitate both rotation and lateral translation of the ultrasound transducer assembly relative to the housing.

In example implementations in which the ultrasound device is configured to facilitate rotation and/or lateral translation of the ultrasound transducer assembly relative to the housing, the ultrasound transducer may take on many different forms without departing from the intended scope of the present disclosure. For example, in some example implementations, the ultrasound transducer may be a single element transducer, while in other example implementations, the ultrasound transducer may be an array of ultrasound elements, such as a linear array, or a one-dimensional or two-dimensional phased array. In one example implementation in which the ultrasound transducer is one-dimensional array (a linear array or phased array), the ultrasound transducer assembly may be moved in angle and/or lateral position to align the axis of the one-dimensional ultrasound array in a direction that is perpendicular to a blood vessel, based on detection and monitoring of Doppler ultrasound signals detected by the array during movement of the ultrasound transducer assembly.

As described above, frictional engagement between the ultrasound transducer assembly and the housing may be provided that is sufficiently low to permit manual actuation of the ultrasound transducer assembly and sufficiently high to maintain the ultrasound transducer assembly in a fixed orientation in the absence of an external applied force.

Additionally or alternatively, a releasable locking means or mechanism may be provided to facilitate the locking of the orientation and/or lateral position of the ultrasound transducer assembly. It will be understood that a wide variety of locking mechanisms may be employed, such as, but not limited to, locking screws/knobs, clamping collars and other clamping mechanisms, latches, and other suitable fixation mechanisms. The locking means may be actuated after determining, based on the detection or monitoring of Doppler ultrasound signals, that the ultrasound transducer is aligned on a vessel of interest.

In some example embodiments, a scale (e.g. an angle scale or a linear position scale with a set of markings) may be provided to facilitate the visual determination of a given angle or position of the ultrasound transducer assembly relative to the housing. For example, the housing and/or the ultrasound transducer assembly may include an orientation scale that permits visual determination of the orientation of the ultrasound transducer relative to the housing, and/or a lateral position scale that permits visual determination of the lateral position of the ultrasound transducer relative to the housing.

While some of the present example embodiments refer to manual positioning of the ultrasound transducer assembly relative to the housing, it will be understood that the positioning may be performed in a manual, autonomous, or semi-autonomous manner. For example, a robotic arm or assembly may be employed to control the angulation and/or positioning of the ultrasound transducer assembly relative to the housing. This may be achieved, for example, using one or more motors operably coupled to the ultrasound transducer assembly, e.g. directly coupled, or coupled through an intermediate actuation mechanism or member extending from the ultrasound transducer assembly.

In some example implementations, an encoding mechanism may be employed to facilitate encoding of the angle and/or lateral position of the ultrasound transducer assembly. For example, an optical encoder or electrical encoder (e.g. conductive or potentiometer based) may be employed. The encoding mechanism may include an encoding interface, such as a reflective interface or conductive interface with a set of markings or conductive features, and an encoding sensor, such as an optical emitter/detector or a conductivity/resistance detection circuit, where the encoding sensor moves relative to the encoding interface, or vice versa. The signal from the encoder sensor may be employed and processed by control and processing circuitry to determine an angle and/or lateral position of the ultrasound transducer assembly relative to the housing.

In another example embodiment, a reference sensor may be employed to facilitate detection of a reference angle and/or reference lateral position of the ultrasound transducer assembly (e.g. a “home” angle and/or lateral position), via detection of a feature, such as an optically reflective or electrically conductive feature. The signal from the reference sensor may be employed and processed by control and processing circuitry to determine when the ultrasound transducer assembly resides at a reference angle and/or a reference lateral position relative to the housing.

It will be understood that the various example embodiments described herein may take on different forms in order to achieve a desired angle between a given vessel and an ultrasound beam generated by the device. For example, as shown in FIG. 12, a distal surface of the housing 710 that faces the patient during use may be angled, relative to a proximal surface of the housing 710 that faces away from the patient during use, so as to apply a compressive force to the skin in the region below the ultrasound elements, at an angle relative to the skin surface, when the device is secured to the patient using an adhesive strip 720 that contacts the proximal surface of the housing 710. As shown in FIG. 12, the ultrasound transducer may be oriented within the housing such that the ultrasound transducer has an emitting surface that is parallel to (or within ±10°, ±15°±20° or ±25° relative to) the distal surface of the housing 710.

Alternatively, as shown in FIG. 13, the ultrasound transducer may be oriented within the housing 710 such that the ultrasound transducer has an emitting surface that is parallel to (or within ±10°, ±15°±20° or ±25° relative to) the proximal surface of the housing 710.

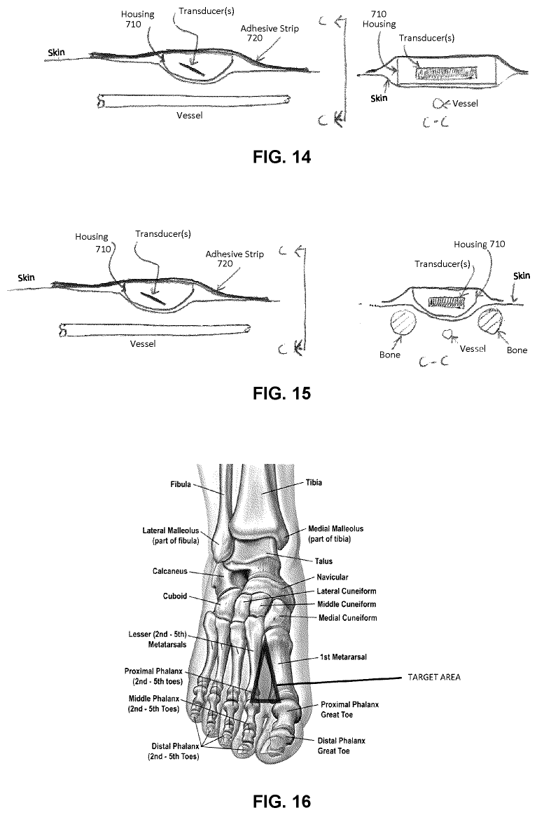

As shown in FIG. 14, the housing 710 may have a planar proximal surface that faces away from the patient during use and a curved distal surface that faces the patient during use. The curved distal surface may have a semi-cylindrical shape, and that the ultrasound transducer may have an emitting surface that is angled relative to the proximal surface of the housing 710. Alternatively, as illustrated by FIG. 15, the distal surface of the housing 710 may have a semi-spherical shape with the transducer angled within the housing. Such a configuration may be advantageous for finding the location when positioning the device between one or more anatomic structures, such as the 1st and 2nd metatarsal bones.

In some example embodiments, a distal (patient-facing) surface of the device, such as a distal surface of the housing 710, or a distal surface of a coupling layer attached to the housing 710, may include one or more anatomical alignment features (e.g. protrusions, recesses or other anatomical indexing structures) that assist in the positioning and/or orienting of the device relative to one or more anatomical structures or features of the patient via a spatial fit (conformal shape matching) between the anatomical alignment features and the one or more anatomical structures or features of the patient.

For example, as shown in FIG. 16, the one or more anatomical structures or features of the patient may include the 1st and 2nd metatarsal bones (used as guideways/landmarks) and the one or more anatomical alignment features may be located on the device to facilitate the alignment of the device with the 1st and 2nd metatarsal bones via the matching of the one or more anatomical alignment features with one or more gaps between the metatarsal bones.

Accordingly, in one example implementation, the device may include a protrusion can be positioned and aligned with the patient anatomy such that that protrusion extends into the soft tissue bounded by the triangular window between the 1st Metatarsal and the 2nd Metatarsal, known as the Cuneiform window, as shown in FIG. 16. A protrusion that extends into the soft tissue in this area will naturally seat the ultrasound transducer between the first and 2nd metatarsals where the Dorsal Metatarsal and Arcuate arteries are located. It is noted that in other example implementations, other positioning mechanisms, such as, but not limited to, a clear window or visual markers on the device, can be employed in the alternative.

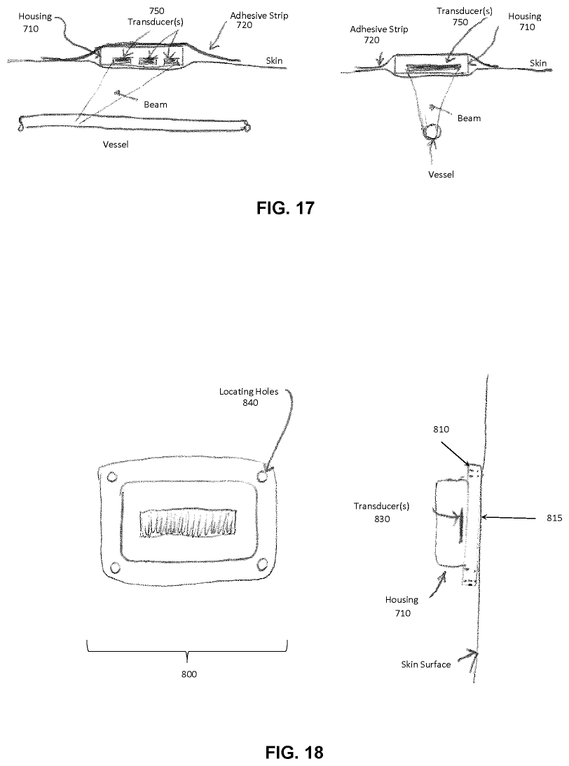

As shown in FIG. 17 (which shows front and side views), the proximal and distal surfaces of the housing 710 may be substantially parallel to the skin surface and house multiple transducers 750 within the housing 710. The transducers 750 may form an interference pattern so as to steer the beam at an angle to the vessel, for example, in order to identify an angle that corresponds to a suitably high Doppler signal (e.g. a Doppler signal that exceeds a pre-selected threshold value). Such an example implementation does not require the transducers to be angled, and as a consequence, the height of the housing can be small relative to the width of the housing. For example, the housing may be 0.5-2.0″ long, 0.5-2.0″ wide, and 0.05-0.2″ high.

In one example embodiment, an ultrasound device 800 is provided that includes a substrate 810 (e.g. a patch) having a bottom surface 815 suitable for attachment to a patient and an upper surface 820 that supports an ultrasound transducer 830 (e.g. a single transducer element or an array of elements), where the ultrasound transducer 830 are supported by the substrate 810 such that when the bottom surface 815 of the substrate 810 is secured to the skin surface, the ultrasound transducer 830 can be operated to deliver ultrasound energy into the patient and receive ultrasound signals. The substrate 810 has one or more holes 840 (apertures) suitable for marking a desired location of the device, as shown in FIG. 18. The ultrasound signals received by device in response to insonification are processed and/or delivered to provide audio Doppler sound and/or velocity waveform to the operator (e.g. via a speaker, graphical user interface, or other suitable medium).

In some example implementations, the operator can move the device over the skin surface while maintaining contact of the bottom surface 815 of the substrate 810 with the skin surface (directly or indirectly through a coupling medium) to search for position of the device that provides a suitable signal. A surgical marker or other marking implement may then be employed to place marks on the skin at the hole locations. The patch can then be removed for later placement, optionally facilitating one or more intervening diagnostic procedures, such as fluoroscopy. When the operator is ready to affix the device to the patient, the device is placed so that the marks line up with the locating holes and an adhesive (e.g. an adhesive material or strip, or other suitable attachment means) is then employed to affix the patch to the patient in the selected location. In another example implementation, the device need not include marking apertures and markings can be made by the operator at a plurality of locations around the periphery of the device substrate or housing. In some example implementations, locating holes can be provided in the central region of the device or between two or more of the transducer elements of a transducer array.

In some example implementations, the ultrasonic transducers are radiopaque and thus block or interrupt the view of the vessels during fluoroscopy. In order to alleviate this issue, an adhesive coupling component 850 may be affixed to a retainer 860 as shown in FIG. 19. The adhesive coupling component 850 and retainer 860 are affixed to the patient's skin. The ultrasound transducer 870 (which may include one or more ultrasound elements) is inserted and retained within the retainer 860 via a suitable attachment mechanism, such as, but not limited to, clips, frictional engagement, and magnets. Fluoroscopic images can be obtained by removing the ultrasound transducer from the retainer. As the retainer 860 and adhesive coupling components 850 are radio-translucent, fluoroscopy images may be obtained while maintaining the retainer 860 and adhesive coupling component 850 secured to the patient. After performing fluoroscopy, the operator can re-attach the transducer to the retainer, thus facilitating the collection of ultrasound Doppler images and/or waveforms in the same position as previously employed (e.g. a position deemed to provide a sufficiently high signal, such as a position associated with the targeting of a particular vessel of interest).

It will be understood that a wide variety of ultrasound devices may be employed according to the embodiments disclosed herein. For example, ultrasound transducers can be formed from piezo-electric, capacitive micromachined ultrasound transducer (CMUT), and polymer CMUT (PolyCMUT) materials. PolyCMUT materials have the advantage of being mechanically flexible, low cost, radio-translucent, optically transparent, light-weight, small, and consume low energy.

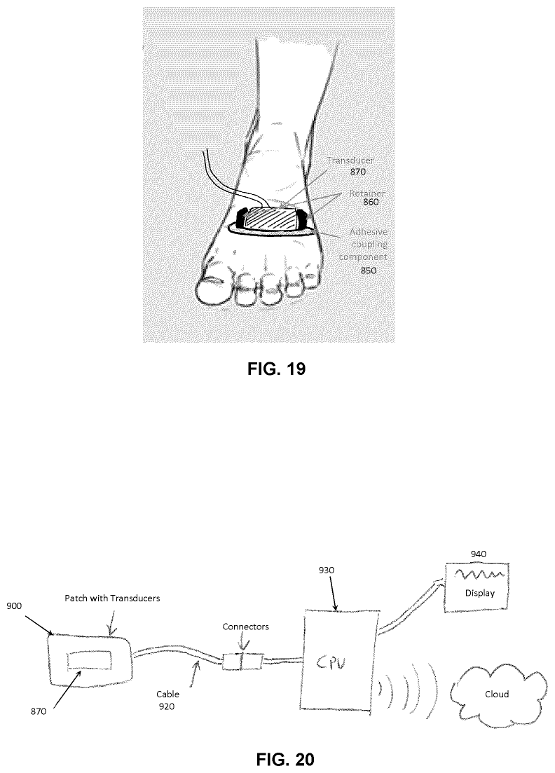

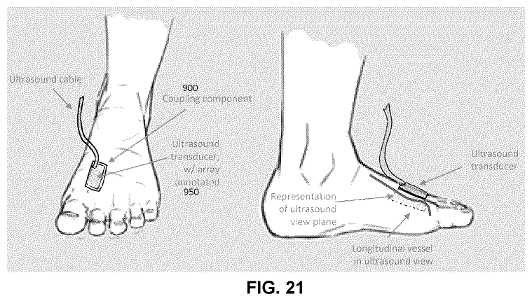

In one example embodiment, shown in FIG. 20, a substrate (patch) 900 supports an ultrasound transducer 910 (e.g. one or more ultrasound elements). The substrate 900 is affixed to the patient. A cable 920 connects the ultrasound transducer to control and processing hardware 930 (which may include components such as those shown in FIG. 6C). A display 940 is connected to the control and processing hardware 930. A user interface may be provided on the display device (e.g. a touch screen) to facilitate various control functions (e.g. gain and beam steering). Alternatively, control functions can be performed via input provided to a separate console. Data (e.g. raw signals or image data, processed images, and or calculated hemodynamic measures) may be transmitted to the cloud (e.g. one or more remote servers) for storage and further analysis. In some example implementations, data is transmitted from the device to the control and processing hardware in wireless form and the device is battery operated. This configuration keeps the patch portion that contacts the patient small and lightweight, reducing errors from movement. In some example implementations, the control and processing hardware is integrated with the device (e.g. supported by the patch or substrate that supports the ultrasound transducer).

FIG. 21 illustrates an example embodiment that includes a substrate 900 (patch) consisting of an ultrasound transducer array 950. The substrate 900 is positioned and affixed to the patient. The ultrasound transducer array 950 is operated as a phased array to generate an ultrasound beam that is transmitted through one or more target vessels (e.g. arteries). The ultrasound beam can be electronically steered and the resulting received ultrasound signals can be employed as feedback to determine a suitable beam angle for the collection of a sufficiently high signal (or signal-to-noise ratio) associated with one or more target vessel(s). Alternatively, an operator can perform these functions via a console that connects to the control and processing hardware. The device may be operated in the absence of the generation of ultrasound images, instead providing a Doppler sound and/or Doppler waveforms corresponding to the selected location of the ultrasound beam that targets one or more vessels. Accordingly, the number of ultrasound elements can be less than that which would conventionally be employed in imaging applications. For example, the number of ultrasound elements may be between 4 and 10, between 4 and 15, between 4 and 20, between 4 and 100, or between 4 and 100. This results in a small, low-cost patch.

While many of the preceding example embodiments employ the use of an adhesive to secure an ultrasound device to a patient, it will be understood that other attachment mechanisms may be employed in the alternative, for example, an elastic band can be used to hold the patch on the patient, optionally in conjunction with adhesive. It will also be understood that while many of the preceding example embodiments refer to the attachment of a substrate or patch to a patient (or subject) this attachment may be direct (i.e. direct contact between the substrate and the patient) or indirect (i.e. contact mediated via in intermediate coupling material or medium, such as ultrasound gel).

While various example embodiments of the present disclosure have been described as being applicable for the measurement of blood flow and hemodynamic measures in the foot, it will be understood that the preceding example embodiments can be employed or adapted to be employed for other anatomical locations, such as, but not limited to, the carotid artery and the hand. For example, Doppler waveforms collected from vessels residing within the hand may be processed to determine a measure such as the “hand acceleration time (HAT)”, which may be clinically useful in the setting of upper extremity arterial disease or steal from arterio-venous fistula.

Various example embodiments of the present disclosure can be employed for remote monitoring and diagnostics. In some example implementations, the device is placed on the patient skin by the patient and data is remotely delivered, optionally in real time, to a remote operator (e.g. clinician) residing at a different physical location that is remote from the patient's home. The remote operator can control one or more of the device parameters (optionally in real time) to facilitate, for example, remote control of beam steering, gain, and other parameters, for example, to obtain a suitably high or optimal signal, and to monitor and/or diagnose a health condition of the patient.

In all embodiments, we can build algorithms to risk stratify the patient based on deep learning analysis on all aspects of the velocity waveform. Embodiments can also have predictive capabilities to guide the physician to either operate on one vessel or not.

As noted above, various example embodiments of the present disclosure can be implemented using ultrasound modalities including, but not limited to, continuous wave, pulsed wave, and/or power Doppler. One or more transducer(s) of the embodiments disclosed herein can be provided in configurations including, but not limited to, a single transducer element, pair of transducer elements, linear array, curvilinear array, or phased array. Any one or more of images, waveforms, processed hemodynamic measures, and/or Doppler audio can be provided to the operator.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. An ultrasound device comprising:
a housing configured for attachment to a skin surface;
an ultrasound transducer assembly supported by said housing, said ultrasound transducer assembly comprising:
an ultrasound transducer; and
a coupling component for coupling ultrasound energy between said ultrasound transducer and the skin surface, wherein said coupling component is in acoustic communication with said ultrasound transducer, and wherein a distal portion of said coupling component is configured to contact and apply pressure to the skin surface when said housing is secured to the skin surface; and
wherein said ultrasound transducer assembly and said housing are configured such that said ultrasound transducer assembly is moveable, relative to said housing, for varying at least one of:
an orientation of said ultrasound transducer assembly relative to said housing; and
a lateral position of said ultrasound transducer assembly relative to said housing;
said ultrasound transducer assembly being supported, relative to said housing, such that a constant pressure is maintained between said distal portion of said coupling component and the skin surface when said ultrasound transducer assembly is moved relative to said housing.

2. The ultrasound device according to claim 1 wherein said housing is configured to permit rotation of said ultrasound transducer assembly about a rotation axis, and wherein a distal surface of said coupling component comprises a circular shape in a plane perpendicular to said rotation axis such that the constant pressure is maintained between said coupling component and the skin surface during rotation of said ultrasound transducer assembly.

3. The ultrasound device according to claim 1 wherein said housing comprises a socket, wherein at least a distal portion of said ultrasound transducer assembly is spherical in shape and resides within said socket, thereby forming a ball and socket joint, said socket comprising a distal aperture through which said ultrasound transducer assembly projects, such that said distal portion of said coupling component contacts and applies pressure to the skin surface when housing is secured to the skin surface, and such that the constant pressure is maintained between said coupling component and the skin surface during rotation of said ultrasound transducer assembly.

4. The ultrasound device according to claim 1 wherein said housing comprises a slot configured to permit lateral translation of said ultrasound transducer assembly relative to said housing in a direction parallel to the skin surface, and such that the constant pressure is maintained between said coupling component and the skin surface during lateral translation of said ultrasound transducer assembly.

5. The ultrasound device according to claim 1 wherein housing is configured to permit lateral translation of said ultrasound transducer assembly relative to said housing in a translation direction that is parallel to the skin surface, and wherein said ultrasound transducer comprises a one-dimensional array of ultrasound elements, the one-dimensional array of ultrasound elements being disposed along a longitudinal axis that is parallel to the translation direction.

6. The ultrasound device according to claim 5 wherein the one-dimensional array of ultrasound elements are angled relative to the skin surface.

7. The ultrasound device according to claim 1 wherein said housing is configured to generate a frictional force as a result of contact with said ultrasound transducer assembly, wherein the frictional force is sufficiently low to permit manual actuation thereof and sufficiently high to maintain said ultrasound transducer assembly in a fixed orientation in the absence of an external applied force.

8. The ultrasound device according to claim 1 wherein said housing is configured to facilitate variation of the orientation of said ultrasound transducer assembly relative to said housing, and wherein at least one said housing and said ultrasound transducer assembly comprises an orientation locking means for releasably locking the orientation of said ultrasound transducer relative to said housing.

9. The ultrasound device according to claim 1 wherein said housing is configured to facilitate variation of the lateral position of said ultrasound transducer assembly relative to said housing, and wherein at least one of said housing and said ultrasound transducer assembly comprises a position locking means for releasably locking the lateral position of said ultrasound transducer relative to said housing.

10. The ultrasound device according to claim 1 wherein said housing is configured to facilitate variation of the orientation of said ultrasound transducer assembly relative to said housing, and wherein at least one of said housing and said ultrasound transducer assembly comprises an orientation scale that permits visual determination of the orientation of said ultrasound transducer relative to said housing.

11. The ultrasound device according to claim 1 wherein said housing is configured to facilitate variation of the lateral position of said ultrasound transducer assembly relative to said housing, and wherein at least one of said housing and said ultrasound transducer assembly comprises a lateral position scale that permits visual determination of the lateral position of said ultrasound transducer relative to said housing.

12. The ultrasound device according to claim 1 further comprising two or more marking apertures that permit marking of the skin surface after securing said housing relative to the skin surface, thereby facilitating re-alignment of said ultrasound device after detachment from the skin surface and re-attachment of said housing to the skin surface.

13. The ultrasound device according to claim 1 further comprising:
at least one motor operably coupled to said ultrasound transducer assembly; and
control circuitry configured to control actuation of said at least one motor for controlling of at least one of the orientation and the lateral position of said ultrasound transducer assembly relative to said housing.

14. The ultrasound device according to claim 13 further comprising an encoding mechanism for encoding at least one of the orientation and the lateral position of said ultrasound transducer assembly relative to said housing, wherein said control circuitry is configured to process a signal from an encoding sensor associated with said encoding mechanism to determine at least one of the orientation and the lateral position of said ultrasound transducer assembly relative to said housing.

15. The ultrasound device according to claim 13 further comprising a reference sensor for detecting at least one of a reference angle and a reference lateral position of said ultrasound transducer assembly relative to said housing, wherein said control circuitry is configured to process a signal from said reference sensor to determine when said ultrasound transducer assembly resides at the reference angle or the reference lateral position.

16. The ultrasound device according to claim 1 wherein said ultrasound transducer assembly and said housing are configured such that said ultrasound transducer assembly is moveable, relative to said housing, for varying the orientation and the lateral position of said ultrasound transducer assembly.

17. The ultrasound device according to claim 1 wherein said ultrasound transducer is a single element ultrasound transducer.

18. The ultrasound device according to claim 1 wherein said ultrasound transducer comprises a plurality of ultrasound transducer elements.

19. The ultrasound device according to claim 1 further comprising a flexible substrate that is attachable to the skin surface for indirectly supporting said housing relative to the skin surface.

20. An ultrasound device comprising:
a housing configured for attachment to a skin surface;
an ultrasound transducer supported relative to said housing such that said ultrasound transducer is moveable, relative to said housing, for varying at least one of:
an orientation of said ultrasound transducer relative to said housing; and
a lateral position of said ultrasound transducer relative to said housing; and
a locking means for releasably locking at least one of the orientation and the lateral position of said ultrasound transducer relative to said housing.

21. An ultrasound device comprising:
a housing configured for attachment to a skin surface;
an ultrasound transducer supported relative to said housing such that said ultrasound transducer is moveable, relative to said housing, for varying at least one of:
an orientation of said ultrasound transducer relative to said housing; and
a lateral position of said ultrasound transducer relative to said housing; and
a scale that permits visual determination of at least one of the orientation and lateral position of said ultrasound transducer relative to said housing.

22. An ultrasound device comprising:
a housing configured for attachment to a skin surface;
an ultrasound transducer assembly supported by said housing, said ultrasound transducer assembly comprising:
an ultrasound transducer; and
a coupling component for coupling ultrasound energy between said ultrasound transducer and the skin surface, wherein said coupling component is in acoustic communication with said ultrasound transducer, and wherein a distal portion of said coupling component is configured to contact and apply pressure to the skin surface when housing is secured to the skin surface; and
wherein said ultrasound transducer assembly and said housing are configured such that said ultrasound transducer assembly is moveable, relative to said housing, for an orientation of said ultrasound transducer assembly relative to said housing;
said ultrasound transducer assembly being supported, relative to said housing, such that a constant pressure is maintained between said distal portion of said coupling component and the skin surface when said ultrasound transducer assembly is moved relative to said housing.

23. An ultrasound device comprising:
a housing configured for attachment to a skin surface;
an ultrasound transducer assembly supported by said housing, said ultrasound transducer assembly comprising:
an ultrasound transducer; and
a coupling component for coupling ultrasound energy between said ultrasound transducer and the skin surface, wherein said coupling component is in acoustic communication with said ultrasound transducer, and wherein a distal portion of said coupling component is configured to contact and apply pressure to the skin surface when housing is secured to the skin surface; and
wherein said ultrasound transducer assembly and said housing are configured such that said ultrasound transducer assembly is moveable, relative to said housing, for varying a lateral position of said ultrasound transducer assembly relative to said housing;
said ultrasound transducer assembly being supported, relative to said housing, such that a constant pressure is maintained between said distal portion of said coupling component and the skin surface when said ultrasound transducer assembly is moved relative to said housing.

24. An ultrasound device comprising:
a housing configured for attachment to a skin surface;
an ultrasound transducer supported relative to said housing such that said ultrasound transducer is moveable, relative to said housing, for varying at least one of:
an orientation of said ultrasound transducer relative to said housing; and
a lateral position of said ultrasound transducer relative to said housing; and
wherein said housing is configured to generate a frictional force as a result of contact with said ultrasound transducer assembly, wherein the frictional force is sufficiently low to permit manual actuation thereof and sufficiently high to maintain said ultrasound transducer assembly in a fixed orientation in the absence of an external applied force.

25. A method of performing Doppler ultrasound, the method comprising:
securing the ultrasound device according to claim 1 to the skin surface of a subject;
while actuating the ultrasound device to vary at least one of the orientation of said ultrasound transducer assembly relative to said housing and the lateral position of said ultrasound transducer assembly relative to said housing:
detecting Doppler ultrasound signals from the ultrasound transducer; and
employing the Doppler ultrasound signals to determine at least one of a suitable orientation and a suitable lateral position of the ultrasound transducer assembly that is associated with detection of blood flow within a blood vessel.

26. The method according to claim 25 wherein the ultrasound device further comprises a locking means for releasably locking at least one of the suitable orientation and the suitable lateral position of said ultrasound transducer relative to said housing, the method further comprising:
after determining at least one of the suitable orientation and the suitable lateral position that is associated with detection of blood flow within the blood vessel, actuating the locking means.

27. The method according to claim 26 wherein said ultrasound transducer assembly and said housing are configured such that said ultrasound transducer assembly is moveable, relative to said housing, for varying both the orientation and lateral position of said ultrasound transducer assembly relative to said housing, and wherein the locking means is configured for releasably locking both the orientation and the lateral position of the ultrasound transducer assembly relative to the frame, the method further comprising:
after varying both the orientation and lateral position of the ultrasound transducer assembly to determine the suitable orientation and the suitable lateral position of the ultrasound transducer that is associated with detection of blood flow within the blood vessel, actuating the locking means to lock both the orientation and the lateral position of the ultrasound transducer assembly.

28. The method according to claim 25 wherein the ultrasound device further comprises a scale that permits visual determination of at least one of the orientation and lateral position of said ultrasound transducer relative to said housing, the method further comprising:
employing the scale to record the at least one of the orientation and lateral position of said ultrasound transducer that is associated with detection of blood flow within the blood vessel.

29. The method according to claim 25 wherein said ultrasound transducer comprises a one-dimensional array of ultrasound elements disposed along a longitudinal axis, and wherein said ultrasound transducer assembly and said housing are configured such that said ultrasound transducer assembly is moveable, relative to said housing, for varying both the orientation and lateral position of said ultrasound transducer assembly relative to said housing, the method further comprising:
employing the Doppler ultrasound signals to determine the suitable orientation and the suitable lateral position of said ultrasound transducer assembly such that said longitudinal axis is substantially perpendicular to an axis of the blood vessel.

30. The method according to claim 25 wherein the housing is indirectly secured to the skin surface via an intermediate flexible substrate adhered to the skin surface.
